(19) United States
(12) Patent Application Publication
(10) Pub. No.: US 2020/0261663 A1
(43) Pub. Date: Aug. 20, 2020

(54) TEMPER EVIDENT INJECTOR LOCK AND A LOCKING METHOD

(71) Applicants: Atilla SEVINCLI, Izmir (TR); ALTERA TIBBI MALZEME SANAYI VE TICARET ANONIM SIRKETI, Izmir (TR)

(72) Inventor: Atilla SEVINCLI, Izmir (TR)

(73) Assignees: Atilla SEVINCLI, Izmir (TR); ALTERA TIBBI MALZEME SANAYI VE TICARET ANONIM SIRKETI, Izmir (TR)

(21) Appl. No.: 16/652,082

(22) PCT Filed: Oct. 2, 2018
(86) PCT No.: PCT/TR2018/050538

§ 371 Date: Mar. 30, 2020

(30) Foreign Application Priority Data
Oct. 5, 2017 (TR) 2017/15048

(51) Int. Cl. A61M 5/50

(52) U.S. Cl. CPC A61M 5/5086

(57) ABSTRACT

A two-stage temper evident injector lock with a security indicator on the two-stage temper evident injector lock and a method of locking the two-stage temper evident injector lock, which has been developed for guaranteeing a security for that a dosage of a fluid contained in the medical injectors (syringes) that are filled with the medications prepared at medication preparation areas, and mostly with specific fluids is not altered before being applied to a subject.

### CROSS REFERENCES TO THE RELATED APPLICATIONS

This application is the national phase entry of International Application No. PCT/TR2018/050538, filed on Oct. 2, 2018, the entire contents of which are incorporated herein by reference.

### TECHNICAL FIELD

The present invention; relates to a two-stage temper evident injector lock with a security indicator thereon and a method of locking said lock, which has been developed for guaranteeing a security so that a dosage of a fluid contained in the medical injectors (syringes) that are filled with the medications prepared at medication preparation areas, and mostly with specific fluids is not altered before being applied to a subject.

### BACKGROUND

In the prior art, several various lock options for preventing any alteration of a dosage of a fluid contained in the medical injectors that are filled with the medications prepared at medication preparation areas, and mostly with specific fluids before use are known. One of said options is simple stopper that only allow a closure for a tip of an injector, however, it is not possible for end users to understand whether the medical injector is unlocked and used before end user uses the injector. These simple stoppers may have different colors and shape. They are suitable to fit in luer groove in the tip of injector to enable a fluid drawn into the syringe to be retained therein. Another lock option is the locks or caps wherein needle in the injector tip is instantly covered after filling, which are uncovered before use. The third option is two-stage locks of which the first stage is removed when turned to an opposite direction after being locked, and an inner stage can be subsequently unlocked.

The lock type that is described in the third option is one of the safest systems currently known in the prior art, which is an invention subject to the patent document numbered U.S. Pat. No. 5,328,474. This lock is formed of two layers. After inner layer is aligned to a grooved tip of an injector, outer layer is turned in a clockwise direction. Thus, that the inner and outer layers are jointly attached and locked to the nozzle of the injector is guaranteed. When turned in an anti-clockwise direction to unlock, the outer layer is removed first by being separated from the inner layer. The inner layer is subsequently turned in anti-clockwise direction and removed. In this manner use of a syringe, whose outer layer of the syringe lock is detached, is avoided as the amount of fluid contained in the syringe may be altered.

Further examples of the other lock types known in the prior art can be included in; the inventions subject to the patent documents numbered US2016310671A1, US20030032923, U.S. Pat. Nos. 8,075,535, 4,571,242, US20080097310, U.S. Pat. Nos. 6,193,688, 4,667,837, 9,402,967B1, U.S. Pat. No. 8,348,895B1, US2011046550A1, U.S. Pat. No. 7,762,988B1, US2010145270A1, WO2005123159A2, U.S. Pat. No. 6,193,688B1 and US2008097310A1.

### SUMMARY

The main aim of the present invention is to guarantee that dosage of the medications and solutions pre-prepared and filled into medical injectors are not altered till they are delivered to end user, and that the injectors are unlocked by end user for the first time after being filled as well as that this can be visually checked by the same end user.

To achieve this aim, the injector lock that has been developed according to the invention has been designed in a two-stage type. After a medication is filled into the injector, the injector and the injector lock are tightly locked by being coupled to each other at their luer lock tips, and the injector lock is turned from its outer stage in clockwise direction. When needed to unlock the injector by turning the outer stage in an anti-clockwise direction, the outer stage of the lock needs to be broken, thereby assuring end user that he/she is the first person to use the injector filled with the medication. In order to use the medication contained in the injector after it is unlocked by breaking its outer stage, its inner portion coupled to the injector via the luer tip has to be removed by being turned in anti-clockwise direction. The inner stage can be attached to the luer tip again, however the outer stage is not able to be coupled to the inner stage again. Therefore, even if the inner stage is attached again at an interval in use, it would be noticed that the injector is used before as the outer stage cannot be attached again. To use the injector lock, it is required either that the medication is filled into the injector by means of needleless adaptors or that needle is removed after filling the medication.

Moreover, in case of any possible trouble or fault in manually locking the invention to the syringe, the injector and the luer lock tip of the injector lock are being coupled to each other and an Allen wrench is inserted in a unidirectional groove, and by turning the former in clockwise direction, the lock according to the invention is enabled to be irreversibly locked to the injector. Even if the Allen wrench is turned in an opposite direction to unlock the lock, the mutual design of a locking end of the Allen wrench and the groove will lead to an idle rotation of the screw and the lock will not be unlocked. The lock can only be removed by unlocking the inner stage after the outer stage is being broken and removed.

### BRIEF DESCRIPTION OF THE DRAWINGS

The drawings that are used for a better understanding of a two-stage temper evident injector lock and a method of locking the same, which has been developed according to the present invention, and the descriptions of these drawings are presented below.

FIG. 1: Shows a bottom view of a two-stage temper evident injector lock according to the invention.

FIG. 2: Shows an isometric, middle-section view of the two-stage temper evident injector lock according to the invention.

FIG. 3: Shows an isometric, vertical-section view of the two-stage temper evident injector lock according to the invention.

FIG. 4: Shows a detail view of a turning part of the two-stage temper evident injector lock according to the invention.

FIG. 5: Shows an isometric, top vertical-section view of the two-stage temper evident injector lock according to the invention.

FIG. 6: Shows a two-dimensional, vertical-section view of the two-stage temper evident injector lock according to the invention.

FIG. 7: Shows an overall view of the two-stage temper evident injector lock according to the invention when used together with an Allen wrench.

FIG. 8: Shows an overall view of the Allen wrench of the two-stage temper evident injector lock according to the invention.

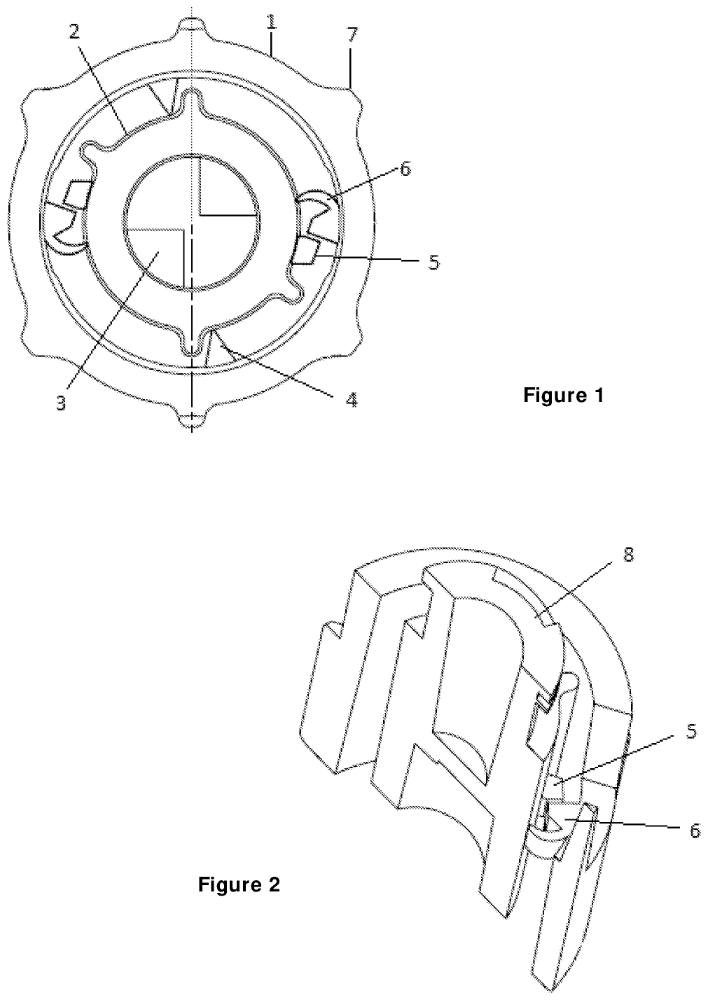

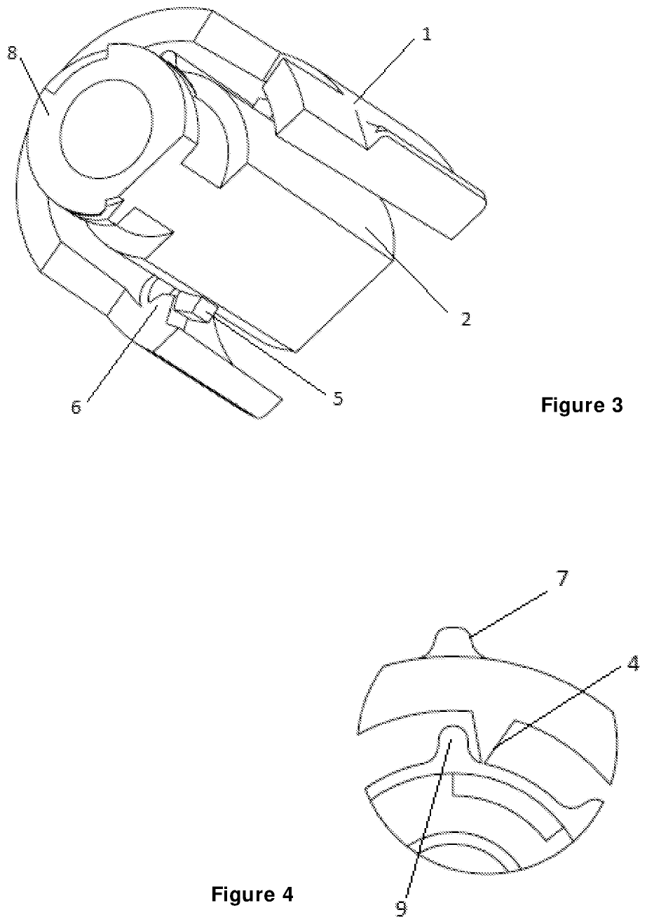

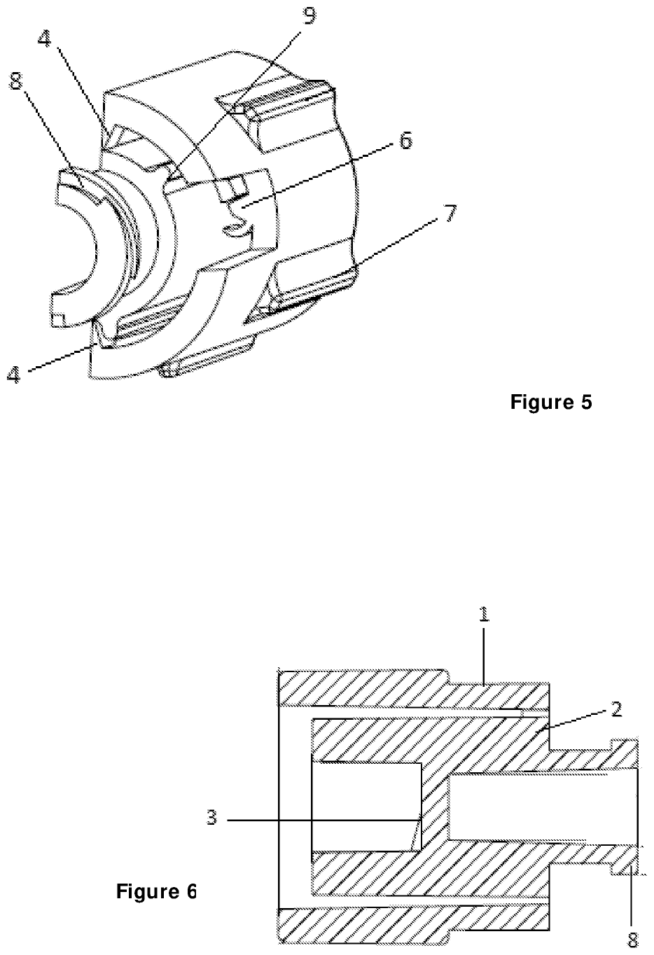

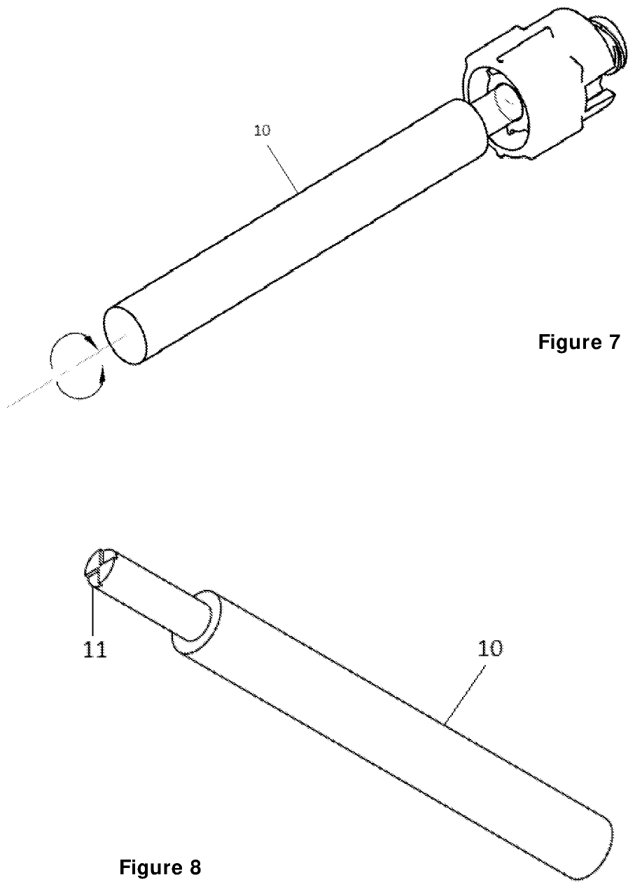

The components and elements shown in the drawings with the aim of better describing the two-stage temper evident injector and the method of locking the same, which has been developed according to the invention, are represented with individual numbers and the related definition of each number is given below.
1. Outer stage
2. Inner stage
3. Unidirectional groove
4. Outer stage upper rotating feather
5. Inner stage lower rotating support
6. Outer stage lower rotating feather
7. Tab support
8. Luer lock
9. Inner stage upper rotating support
10. Allen wrench
11. Allen wrench locking end

### DETAILED DESCRIPTION OF THE EMBODIMENTS

Processes of locking and unlocking in a two-stage temper evident injector lock are performed as described below, wherein said two-stage temper evident injector lock has thereon a plurality of tab supports (7), an outer stage lower rotating feather (6) and an outer stage upper rotating feather (4), being coupled to an outer stage (1) and an inner stage (2) at an end of the outer stage lower rotating feather (6) in a comma shape, and; it has also thereon a unidirectional groove (3), a luer lock (8), an inner stage upper rotating support (9) and an inner stage lower rotating support (5).

In the two-stage temper evident injector lock wherein an outer stage (1) and an inner stage (2) are only coupled to each other at an end of an outer stage lower rotating feather (6) with a comma shape, in order to lock the injector which a medication is filled, the outer stage (1) is grasped and aligned to a luer lock tip of the injector so as to correspond to a luer lock (8) part of the lock, and then is turned in clockwise direction being backed by a tab support (7). During this rotation, with a force obtained from the tabs, the outer stage lower rotating feather (6) pushes the inner stage lower rotating support (5) whilst the outer stage upper rotating feather (4) pushes the inner stage upper rotating support (9) in clockwise direction, which allows the luer lock (8) to be inserted in the injector luer lock tip and locked therein.

No matter how much force is applied during this rotational movement, as no force corresponds to the comma shaped part of the outer stage lower rotating feather (6), it is not possible to break this part and the inner stage (2) and the outer stage (1) are not separated from each other.

Two steps are required to unlock the locked injector that has been delivered to end user. In the first step, the outer stage (1) is turned in anti-clockwise direction with the support of the tab support (7). With this rotational movement, a certain amount of force is applied to the comma shaped part of the outer stage lower rotating feather (6), and the outer stage (1) and the inner stage (2) are disconnected. Such disconnection can be detected with a breaking sound.

The outer stage (1) that has been disconnected is drawn in an opposite direction to the injector and pulled out. In the second step, the inner stage (2) is turned in anti-clockwise direction being backed by the inner stage upper rotating support (9), which enables the luer lock (8) to be detached from the injector luer lock tip.

In the method of locking that is used in case of any possible trouble or fault in manually locking; after the luer lock (8) is aligned to the injector luer lock tip so as to corresponds to the later, a flat-ended Allen wrench (10) is attached to a unidirectional groove (3) via an Allen wrench locking end (11) at an end of the former, and is turned in clock-wise direction, which ensures an irreversibly locking for the lock to the injector in return.

The invention, thanks to these characteristics, is capable of being made in a body in an injection mold and manually coupled to the injector (syringe) by being turned in clock-wise direction from the outer stage (1), and also breaking its outer stage (1) in a manual manner and by being turned in anti-clockwise direction from the outer stage (1) as well as pulling its inner stage (2) out of the syringe by manually being turned in anti-clockwise direction.

Even if the Allen wrench (10) is turned in an opposite direction to unlock the lock, the mutual design of the Allen wrench locking end (11) and the unidirectional groove (3) will lead to an idle rotation of the screw and the lock will not be unlocked. The lock can only be pulled out by unlocking the inner stage (2) after the outer stage (1) is being broken and removed.

The invention is achieved by performing the steps of coupling the outer stage (1) and the inner stage (2) only at the comma shaped end of the outer stage lower rotating feather (6), allowing the luer lock (8) to be inserted in the injector luer lock nozzle and to be locked therein once the outer stage lower rotating feather (6) pushes the inner stage lower rotating support (5) whilst the outer stage upper rotating feather (4) pushes the inner stage upper rotating support (9) in clockwise direction with the force obtained from the tabs so as to lock the injector lock, during this rotational movement, not breaking the comma shaped part of the outer stage lower rotating feather (6) and not detaching the inner stage (2) and the outer stage (1) from each other no matter how much force is applied, turning the outer stage (1) in anti-clockwise direction with the support of the locking and unlocking tab support (7) in order to unlock the injector, applying a certain amount of force to the comma shaped part of the outer stage lower rotating feather (6) with this rotational movement and disconnecting the outer stage (1) and the inner stage (2), removing the outer stage (1) and turning the inner stage (2) in anti-clockwise direction with the support of the inner stage upper rotating support (9) and thereby separating the luer lock (8) from the injector luer lock nozzle.

What is claimed is:

1. A two-stage temper evident injector lock having a plurality of tab supports the two-stage temper evident injector lock, comprising an outer stage with an outer stage upper rotating feather and an outer stage lower rotating feather at an inner section of the outer stage and the inner stage, being coupled to the outer stage at an end of the outer stage lower rotating feather having a comma shape, also having on the two-stage temper evident injector lock a unidirectional groove, a luer lock, an inner stage upper rotating support, and an inner stage lower rotating support, wherein the two-stage temper evident injector lock comprises the following in order for a lock to be secured to an injector in case difficulty or problems are experienced during manual locking;
an Allen wrench that comprises a flat Allen wrench locking end at an end portion of the Allen wrench, and
the unidirectional groove on the lock, wherein the unidirectional groove corresponds to the Allen wrench locking end.

2. A method of applying the two-stage temper evident injector lock according to claim 1, comprising the process steps of;
coupling the outer stage and the inner stage only at a comma shaped end of the outer stage lower rotating feather,
allowing the luer lock to be inserted in an injector luer lock tip and to be locked in the injector luer lock tip once the outer stage lower rotating feather pushes the inner stage lower rotating support whilst the outer stage upper rotating feather pushes the inner stage upper rotating support in clockwise direction with force obtained from a plurality of tabs so as to lock the two-stage temper evident injector lock,
turning the outer stage in anti-clockwise direction with a support of a locking and unlocking tab support in order to unlock the injector, applying force to a comma shaped part of the outer stage lower rotating feather with rotational movement of turning the outer stage in anti-clockwise direction and disconnecting the outer stage and the inner stage, removing the outer stage and turning the inner stage in anti-clockwise direction with a support of the inner stage upper rotating support and by the support of the inner stage upper rotating support separating the luer lock from the injector luer lock tip.

3. The two-stage temper evident injector lock according to claim 1; comprising a configuration, wherein in the configuration even if the Allen wrench is turned in counter clockwise direction to unlock the lock, a mutual design of the Allen wrench locking end and the unidirectional groove leads to an idle rotation of a screw.

4. A method of locking the two-stage temper evident injector lock that is used in case of any possible difficulty or problems experienced during manually locking of the lock according to claim 1, comprising the steps of;
corresponding the luer lock to the injector luer lock tip,
attaching a flat-ended Allen wrench to the unidirectional groove via the Allen wrench locking end at the end portion of the flat-ended Allen wrench, and
irreversibly locking the lock to the injector by turning the Allen wrench in clock-wise direction.
